(19) United States
(12) Patent Application Publication
(10) Pub. No.: US 2019/0040476 A1
(43) Pub. Date: Feb. 7, 2019

(54) RECOMBINANT MICROORGANISM OF GENUS KOMAGATAEIBACTER, METHOD OF PRODUCING CELLULOSE BY USING THE SAME, AND METHOD OF PRODUCING THE MICROORGANISM

(71) Applicant: Samsung Electronics Co., Ltd., Suwon-si (KR)

(72) Inventors: Soonchun Chung, Seoul (KR); Sunghaeng Lee, Seoul (KR); Jinhwan Park, Suwon-si (KR)

(21) Appl. No.: 16/052,056

(22) Filed: Aug. 1, 2018

(30) Foreign Application Priority Data
Aug. 2, 2017 (KR) 10-2017-0098071
Nov. 29, 2017 (KR) 10-2017-0161834

(51) Int. Cl. C12R 1/01; C12N 9/12; C12N 1/20; C08L 1/02

(52) U.S. Cl. CPC C12R 1/01; C12N 9/1229; C12Y 207/04001; C08L 1/02; C12N 1/20

(57) ABSTRACT

Provided is a microorganism of the genus Komagataeibacter having enhanced cellulose productivity and yield, a method of producing cellulose by using the same, and a method of producing the microorganism.

### CROSS-REFERENCE TO RELATED APPLICATION

This application claims the benefits of Korean Patent Application No. 10-2017-0098071, filed on Aug. 2, 2017, and Korean Patent Application No. 10-2017-0161834, filed on Nov. 29, 2017, in the Korean Intellectual Property Office, the entire disclosures of which are hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 36,684 Byte ASCII (Text) file named “737735_ST25.TXT,” created on Aug. 1, 2018.

### BACKGROUND

### 1. Field

The present disclosure relates to a recombinant microorganism of the genus Komagataeibacter comprising a genetic modification that increases expression of polyphosphate kinase (PPK) or an activity thereof, a method of producing cellulose by using the recombinant microorganism, and a method of producing the recombinant microorganism.

### 2. Description of the Related Art

Cellulose produced by culturing microorganisms, also known as “biocellulose” or “microbial cellulose,” exists as a primary structure of β-1,4 glucan composed of glucose that forms a network structure of fibril bundles. Microbial cellulose is typically about 100 nm or less in width, and, unlike plant cellulose, is free of lignin or hemicellulose. Additionally, compared to plant cellulose, microbial cellulose has increased water absorption and retention capacity, higher tensile strength, higher elasticity, and higher heat resistance. Due to these characteristics, microbial cellulose has been developed for applications in a variety of fields, such as cosmetics, medical products, dietary fibers, audio speaker diaphragms, and functional films.

Therefore, there is a need to develop new microorganisms and methods to increase the production of microbial cellulose.

### SUMMARY

Provided is a recombinant microorganism of the genus Komagataeibacter comprising a genetic modification that increases expression or activity of of polyphosphate kinase (PPK).

Also provided is a method of producing cellulose by using the recombinant microorganism, as well as a method of producing the recombinant microorganism.

### BRIEF DESCRIPTION OF THE DRAWINGS

These and/or other aspects will become apparent and more readily appreciated from the following description of the embodiments, taken in conjunction with the accompanying drawings in which:

FIG. 1 shows cellulose nanofiber (CNF) production of a K. xylinus strain into which a pfkA gene is introduced compared to control strains;

FIG. 2 shows CNF yield of a K. xylinus strain into which a pfkA gene is introduced compared to control strains;

FIG. 3 shows CNF production and yield of a K. xylinus strain into which a pfkA gene is introduced during fermentation in a medium free of carboxy methyl cellulose (CMC) compared to control strains;

FIG. 4 shows CNF production and yield of a K. xylinus strain into which a pfkA gene is introduced during fermentation in a medium including CMC compared to a control strain; and

FIG. 5 shows results of comparing CNF production of SK3 strains, in which the SK3 strains are introduced with the indicated ppk gene.

### DETAILED DESCRIPTION

The term “increase in expression”, as used herein, may refer to an increase in transcription or translation of a gene encoding a protein or an enzyme.

The term “increase in activity” or “increased activity”, or like terms, as used herein refers to a detectable increase in an activity level of a modified (e.g., genetically engineered) cell, protein, or enzyme compared to a cell, protein, or enzyme of the same type that does not have the given genetic modification (e.g., a parent cell or a native, original, or “wild-type” cell, protein, or enzyme). For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more relative to the activity of a non-engineered cell (e.g., parent cell), protein, or enzyme of the same type A cell having an increased activity of a protein or an enzyme may be identified by using any method known in the art.

A cell having increased activity of an enzyme or a polypeptide may be achieved by an increase in expression or specific activity of the enzyme or polypeptide. The increase in expression may be caused by introduction of a polynucleotide encoding the enzyme or the polypeptide into a cell, by otherwise increasing the copy number of the polynucleotide that encodes the enzyme or polypeptide, or by modification of a regulatory region of the polynucleotide encoding the enzyme or polypeptide so as to increase expression thereof. The introduction of a polynucleotide encoding the enzyme or polypeptide may be a transient introduction in which the gene is not integrated into a genome, or an introduction that results in integration of the gene into the genome. The introduction may be performed, for example, by introducing a vector comprising a polynucleotide encoding the enzyme or polypeptide into the cell.

A polynucleotide introduced into the cell may be operably linked to a regulatory sequence that allows expression of the enzyme or polypeptide, for example, a promoter, a polyadenylation site, or a combination thereof. When an exogenous polynucleotide is introduced, the polynucleotide may comprise a sequence that is endogenous or heterologous to the microorganism in which it is inserted. As used herein, an endogenous gene refers to a polynucleotide that is present in the intrinsic genetic material of the microorganism prior to a given genetic manipulation, for instance, a polynucleotide present in the genetic material of the wild-type or native microorganism. The term “heterologous” means “foreign” or “not native” to the species.

An increase in copy number of a polynucleotide refers to any increase in copy number. For example, an increase in copy number may be caused by introduction of an exogenous polynucleotide (whether endogenous or heterologous) or amplification of an endogenous polynucleotide. In one embodiment, an increase in copy number may be achieved by genetically engineering a cell so that the cell has a polynucleotide that does not exist in a non-engineered cell. The introduction of a polynucleotide may be a transient introduction in which the polynucleotide is not integrated into a genome, or an introduction that results in integration of the polynucleotide into the genome. The introduction may be performed, for example, by introducing a vector into the cell, the vector including a polynucleotide encoding a target polypeptide, and then, replicating the vector in the cell, or by integrating the polynucleotide into the genome.

The introduction of the gene may be performed via a known method, for example, transformation, transfection, or electroporation.

The term “vector” or “vehicle”, as used herein, refers to a nucleic acid molecule that is able to deliver nucleic acids linked thereto into a cell. The vector may include, for example, a plasmid expression vector, a virus expression vector, such as a replication-defective retrovirus, adenovirus, or an adeno-associated virus.

The genetic modification used in the present disclosure may be performed by any molecular biological method known in the art.

The term “parent cell” refers to an original cell, for example, a non-genetically engineered cell of the same type as an engineered microorganism. With respect to a particular genetic modification, the “parent cell” may be a cell that lacks the particular genetic modification, but is identical in all other respects. Thus, the parent cell may be a cell that is used as a starting material to produce a genetically engineered microorganism having an increased activity of a given protein (e.g., a protein having a sequence identity of about 90% or higher with respect to phosphofructose kinase).

The term “gene” and “polynucleotide”, as used herein are synonymous and refers to a nucleic acid fragment encoding a particular protein, and may optionally include a regulatory sequence of a 5′-non coding sequence and/or a 3′-non coding sequence.

The term “sequence identity” of a polynucleotide or a polypeptide, as used herein, refers to a degree of identity between bases or amino acid residues of sequences obtained after the sequences are aligned so as to best match in certain comparable regions. The sequence identity is a value that is measured by comparing two sequences in certain comparable regions via optimal alignment of the two sequences, in which portions of the sequences in the certain comparable regions may be added or deleted compared to reference sequences. A percentage of sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (that is, the size of a range), and multiplying a result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), MegAlign™ (DNASTAR Inc), etc.

Various levels of sequence identity may be used to identify various types of polypeptides or polynucleotides having the same or similar functions or activities. For example, the sequence identity may include a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

The term “genetic modification”, as used herein, refers to an artificial alteration in a constitution or structure of a genetic material of a cell.

An aspect of the present disclosure provides a recombinant microorganism including a genetic modification that increases phosphofructose kinase (PPK) activity. Polyphosphate kinase (PPK) is an enzyme that catalyzes a reversible reaction of converting NTP+(phosphate)n to NDP+(phosphate)n+1. Here, NTP and NDP represent nucleoside triphosphate and nucleoside diphosphate, respectively. NTP may be ATP, GTP, CTP, or UTP. NDP may be ADP, GDP, CDP, or UDP. In an embodiment, PPK catalyzes both the forward and reverse reactions, and may have higher catalytic activity for the reverse reaction than catalytic activity for the forward reaction than the reverse reaction. In another embodiment, PPK may have higher catalytic activity for conversion of NDP to NTP in a reaction using GDP, CDP, UDP, or a combination thereof as the substrate, compared to using ADP as the substrate. That is, PPK may convert NDP to NTP by using inorganic polyphosphate as a donor. NTP, particularly, UTP may be used in the synthesis of cellulose. PPK may have high catalytic activity for conversion of NDP to NTP in a reaction using UDP, GDP, or UDP and GDP as a substrate, compared to using ADP, in the reverse reaction. PPK may have the highest activity and selectivity for pyrimidine nucleoside diphosphate. This activity is also called polyphosphate-driven nucleoside diphosphate kinase (PNDK) activity. This activity may be measured by incubating a reaction mixture including 75 mM polyphosphate (as phosphate), 30 mM MgCl₂, 5 mM NDP, and 50 mM Tris-HCl (pH 7.8) at 30° C.

PPK may be heterologous or endogenous to the microorganism. The PPK may be an enzyme classified as EC 2.7.4.1. PPK may be from bacteria. For instance, PPK may be from the genus Silicibacter or the genus Rhodobacterales. More specficially, PPK may be from Silicibacter pomeroyi, Silicibacter lacuscaerulensi, or Rhodobacterales bacterium.

In an embodiment, the genetic modification that increases PPK activity, is the introduction of a polynucleotide encoding a PPK having an activity belonging to EC 2.7.4.1.

In another embodiment the genetic modification that increases PPK activity is the introduction of a polynucleotide encoding a polypeptide comprising an amino acid sequence having a sequence identity of about 90% or higher, about 95% or higher, or about 100% to the amino acid sequence of SEQ ID NO: 44, 46, or 48. For example, a polynucleotide comprising a nucleotide sequences having a sequence identity of about 90% or higher, about 95% or higher, or about 100% to the nucleotide sequence of SEQ ID NO: 45, 47, or 49.

The microorganism comprising the genetic modification that increases PPK activity may have enhanced cellulose productivity as compared to a microorganism of the same type without the genetic modification (e.g., as compared to a parent microorganism). The cellulose may also be called nanocellulose, cellulose nanofiber (CNF), microfibrillated cellulose (MFC), nanocrystalline cellulose (NCC), or bacterial nanocellulose. The cellulose may be cellulose free of lignin or hemicelluloses. A fiber width of the cellulose may be about 100 nm or less, about 90 nm or less, about 80 nm or less, about 70 nm or less, about 60 nm or less, about 50 nm or less, about 40 nm or less, about 30 nm or less, about 20 nm or less, or about 10 nm or less. The cellulose may have high absorbency, high strength, high elasticity, high heat resistance or a combination thereof.

The recombinant microorganism of the invention may further include a genetic modification that increases phosphofructose kinase (PFK) activity.

PFK is a protein that phosphorylates fructose-6-phosphate into fructose-1,6-bisphosphate in glycolysis. PFK may catalyze conversion of ATP and fructose-6-phosphate into fructose-1,6-bisphosphate and ADP. PFK is allosterically activated by ADP and diphosphonucleoside, and allosterically inhibited by phosphoenolpyruvate. The PFK may be heterologous or endogenous to the modified microorganism.

PFK may be PFK1 (also called “PFKA”). PFK1 may belong to the enzyme classified as EC 2.7.1.11. PFK may be derived from bacteria. PFK may be derived from the genus Escherichia, the genus Bacillus, the genus Mycobacterium, the genus Zymomonas, or the genus Vibrio. For example, PFK may be derived from E. coli , such as E. coli MG1655.

In an embodiment, the genetic modification that increases PFK activity in the recombinant microorganism is the introduction of a gene encoding a PFK having an activity belonging to EC 2.7.1.11.

In another embodiment, the genetic modification that increases PFK activity in the recombinant microorganism is the introduction of a gene encoding a polypeptide comprising an amino acid sequence having a sequence identity of about 90% or higher, about 95% or higher, or about 100% with the amino acid sequence of SEQ ID NO: 1. For example, the gene encoding PFK may comprise a nucleotide sequence having a sequence identity of about 90% or higher, about 95% or higher, or about 100% with the nucleotide sequence of SEQ ID NO: 2.

In the microorganism, the genetic modification may be one or more of increase in the expression of the gene encoding PFK and increase in the expression of the gene encoding PPK. The genetic modification may be an increase of the copy number of the gene encoding PFK or a modification of an expression regulatory sequence of the gene encoding PFK. Further, the genetic modification may be an increase of the copy number of the gene encoding PPK or a modification of an expression regulatory sequence of the gene encoding PFK. The increase of the copy number may be caused by introduction of the gene into a cell from the outside or by amplification of an endogenous gene.

The genetic modification may introduce the gene encoding PFK and the gene encoding PPK, for example, via a vehicle such as a vector. One or more of the gene encoding PFK and the gene encoding PPK may exist within or outside the chromosome. Furthermore, a plurality of genes (e.g., a plurality of copies) encoding PFK and/or genes encoding PPK may be introduced, for example, 2 or more, 5 or more, 10 or more, 30 or more, 50 or more, 100 or more, or 1000 or more of each of a gene encoding PFK or PPK.

The recombinant microorganism may belong to the genus Komagataeibacter, Gluconacetobacter, or Enterobacter, and may produce bacterial cellulose.

In one embodiment, the microorganism may belong to the genus Komagataeibacter, such as K. xylinus (also, referred to as “G. xylinus”), K. rhaeticus, K. swingsii, K. kombuchae, K. nataicola, or K. sucrofermentans. In some embodiments, the microorganism may have bacterial cellulose productivity. In some embodiments, the microorganism may not have endogenous PFK1. In some embodiments, the microorganism may not have an endogenous glycolytic pathway. Also, in some embodiments, the microorganism may not have endogenous PPK, or may not endogenously have PPK having higher catalytic activity for a reverse reaction than for a forward reaction in the reversible reaction catalyzed by the PPK enzyme. Further, the microorganism may not endogenously have PPK having high catalytic activity for conversion of NDP to NTP in a reaction using GDP, CDP, or UDP as a substrate, compared to using ADP, in the reverse reaction.

Another aspect of the invention provides a method of producing cellulose, the method including culturing a recombinant microorganism in a medium to produce cellulose; and collecting the cellulose from a culture.

In the inventive method, the recombinant microorganism may be any microorganism described herein.

In an embodiment the method comprises culturing a recombinant microorganism including a genetic modification that increases a polyphosphate kinase activity in a medium to produce cellulose; and collecting the cellulose from a culture. In another embodiment the method comprises culturing a recombinant microorganism including a genetic modification that increases a polyphosphate kinase activity and a genetic modification that increases expression of phosphofructose kinase or an activity thereof in a medium to produce cellulose; and collecting the cellulose from a culture.

The culturing may be performed in a medium containing a carbon source, for example, glucose. The medium used for culturing the microorganism may be any general medium suitable for host cell growth, such as a minimal or complex medium containing appropriate supplements. The suitable medium may be commercially available or prepared by a known preparation method.

The medium may be a medium that may satisfy the requirements of a particular microorganism depending on a selected product of culturing. The medium may be a medium including components selected from the group consisting of a carbon source, a nitrogen source, a salt, trace elements, and combinations thereof.

The medium may include ethanol or cellulose (e.g., exogenous or “added” cellulose, as distinguished from cellulose produced by the microorganism being cultured). The ethanol may be about 0.1%(v/v) to 5%(v/v), for example, about 0.3%(v/v) to 2.5%(v/v), about 0.3%(v/v) to 2.0%(v/v), about 0.3%(v/v) to 1.5%(v/v), about 0.3%(v/v) to 1.25%(v/v), about 0.3%(v/v) to 1.0%(v/v), about 0.3%(v/v) to 0.7%(v/v), or about 0.5%(v/v) to 3.0%(v/v) with respect to a volume of the medium. The cellulose may be about 0.5%(v/v) to 5%(w/v), about 0.5%(v/v) to 2.5%(w/v), about 0.5%(v/v) to 1.5%(w/v), or about 0.7%(v/v) to 1.25%(w/v) with respect to a weight of the medium. The cellulose may be carboxylated cellulose. The cellulose may be carboxy alkyl cellulose. The cellulose may be carboxy methyl cellulose (CMC). The CMC may be sodium CMC.

The culturing conditions may be appropriately controlled for the production of a selected product, for example, cellulose. The culturing may be performed under aerobic conditions for cell proliferation. The culturing may be performed by spinner culture or static culture without shaking. A density of the microorganism may be a density which gives enough space so as not to disturb production of cellulose.

The term “culture conditions”, as used herein, mean conditions for culturing the microorganism. Such culture conditions may include, for example, a carbon source, a nitrogen source, or an oxygen condition utilized by the microorganism. The carbon source that may be utilized by the microorganism may include monosaccharides, disaccharides, or polysaccharides. The carbon source may include glucose, fructose, mannose, or galactose as an assimilable glucose. The nitrogen source may be an organic nitrogen compound or an inorganic nitrogen compound. The nitrogen source may be exemplified by amino acids, amides, amines, nitrates, or ammonium salts. An oxygen condition for culturing the microorganism may be an aerobic condition of a normal oxygen partial pressure or a low-oxygen condition including about 0.1° A to about 10% oxygen in the atmosphere. A metabolic pathway may be modified in accordance with a carbon source or a nitrogen source that may be actually used by a microorganism.

The method may include collecting the cellulose from the culture. The separating may be, for example, collecting of a cellulose pellicle which is formed on the top of the medium. The cellulose pellicle may be collected by physically stripping off the cellulose pellicle or by removing the medium. The separating may be collecting of the cellulose pellicle while maintaining its shape without damage.

Still another aspect provides a method of producing the recombinant microorganism having enhanced cellulose productivity, the method including introducing the gene encoding polyphosphate kinase into a microorganism having cellulose productivity. The method may further include introducing the gene encoding PFK into the microorganism.

The introducing of the gene may be introducing of a vehicle including the gene into the microorganism. In the method, the genetic modification may include amplifying the gene, engineering a regulatory sequence of the gene, or engineering a sequence of the gene itself. The engineering may be insertion, substitution, conversion, or addition of a nucleotide.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

Hereinafter, the present invention will be described in more detail with reference to Examples. However, these Examples are for illustrative purposes only, and the scope of the invention is not intended to be limited by these Examples.

### EXAMPLE 1

### Preparation of K. xylinus Including Phosphofructose Kinase Gene and Production of Cellulose

In this Example, Komagataeibacter xylinus DSM2325 was introduced with an exogenous PFK gene, and the microorganism introduced with the gene was cultured so that the microorganism was allowed to consume glucose and produce cellulose, thereby examining effects of the gene introduction on cellulose productivity.

1. Preparation of Vector for Over-Expressing pfkA

The phosphofructose kinase (pfk) gene in K. xylinus was introduced by homologous recombination. A specific procedure is as follows.

An amplification product was obtained by PCR amplification using a pTSa-EX1 vector (SEQ ID NO: 9) as a template and a set of primers of SEQ ID NO: 5 and SEQ ID NO: 6 and a set of primers of SEQ ID NO: 7 and SEQ ID NO: 8. The amplification product was cloned by using an In-Fusion GD cloning kit (Takara) at the BamHl and Sall restriction sites of the pTSa-EX1 vector. The pTSa-EX1 vector is a shuttle vector which is replicable in both E. coli and X. xylinus.

In order to introduce pfkA by homologous recombination, an open reading frame (ORF) (SEQ ID NO: 2) of the pfkA gene was produced by PCR amplification using a genome DNA of E. coli K12 MG1655 as a template and a set of primers of SEQ ID NO: 3 and SEQ ID NO: 4 as primers. Fragments of the pfkA gene were cloned at the BamHI and Sall restriction enzyme sites of the pTSa-EX11 vector by using an In-Fusion GD cloning kit (Takara) to prepare vector pTSa-Ec.pfkA for over-expressing pfkA.

2. Preparation of Vector for Inserting E. coli pfkA Gene

A tetA gene was amplified by PCR amplification using a pTSa-Ec.pfkA vector as a template and a set of primers of SEQ ID NO: 10 and SEQ ID NO: 11 as primers. The PCR product was cloned at an EcoRI restriction enzyme site of a pMSK+ vector (Genbank Accession No. KJ922019) by using an In-fusion GD cloning kit (Takara) to prepare a pTSK+ vector.

A homologous region of a site to which a pfkA gene was about to be inserted was amplified by PCR using a genome DNA of K. xylinus as a template and each of primer sets of SEQ ID NOS: 12 and 13, SEQ ID NOS: 14 and 15, and SEQ ID NOS: 16 and 17 as primers, and the amplification product was cloned at an EcoRI restriction enzyme site of a pTSK+ vector by using an In-fusion GD cloning kit (Takara) to prepare a pTSK-(del)2760 vector. 2760 gene encodes cytoplasmic NADPH-dependent glucose dehydrogenase.

A Ptac::Ec.pfkA gene was amplified by PCR amplification using the pTSa-Ec.pfkA vector as a template and a primer set of SEQ ID NO: 18 and SEQ ID NO: 19 as primers. The PCR product was cloned at an EcoRI restriction enzyme site of a pTSK-(del)2760 vector by using an In-fusion GD cloning kit (Takara) to prepare a pTSK-(del)2760-Ec.pfkA vector.

3. Introduction of Phosphofructose Kinase Gene

In order to introduce the nucleotide sequence of SEQ ID NO: 2, which is a pkfA gene of E. coli, to K. xylinus, a cassette for inserting a Ptac::Ec.pfkA gene was amplified using the pTSK-(del)2760-Ec.pfkA vector as a template and a primer set of SEQ ID NO: 12 and SEQ ID NO: 17 as primers, and the amplification product was introduced into a K. xylinus strain by the following transformation method. A specific procedure is as follows.

The K. xylinus strain was spread on an HS medium (0.5% peptone, 0.5% yeast extract, 0.27% Na₂HPO₄, 0.15% citric acid, 2% glucose, and 1.5% bacto-agar) supplemented with 2% glucose, and then cultured at 30° C. 3 days. The strain was inoculated in 5 ml of HS medium supplemented with 0.2%(v/v) of cellulase (sigma, Cellulase from Trichoderma reesei ATCC 26921), and then cultured at 30° C. 2 days. A cell suspension thus cultured was inoculated in 100 ml of HS medium supplemented with 0.2%(v/v) of cellulase so that cell density (OD₆₀₀) was 0.04, and then the resultant was cultured at 30° C. so that cell density was 0.4 to 0.7. The cultured strain was washed with 1 mM of HEPES buffer, washed three times with 15% glycerol, and re-suspended with 1 ml of 15% glycerol to prepare competent cells.

100 μl of the competent cells thus prepared was transferred to a 2 mm electro-cuvette, 3 μg of the Ptac::Ec.pfkA cassette prepared in the clause 2 was added thereto, and a vector was introduced into the competent cells by electroporation (2.4 kV, 200 Ω, 25 μF). The vector-introduced cells were re-suspended in 1 ml of HS medium containing 2% glucose and 0.1%(v/v) cellulase, transferred to a 14 ml round-bottom tube, and cultured at 30° C. and 160 rpm for 16 hours. The cultured cells were spread on HS medium supplemented with 2% glucose, 1% ethanol, and 5 pg/ml of tetracycline, and cultured at 30° C. for 4 days. Strains having a tetracycline resistance were selected to prepare pfk gene-over-expressing strains.

4. Glucose Consumption and Cellulose and Gluconate Productions

The designated K. xylinus strains were inoculated into 50 ml of HS medium supplemented with 5% glucose and 1% ethanol, and the resultant was cultured under stirring at 30° C. at 230 rpm for 5 days. Then, glucose consumption and cellulose production were quantified. Glucose and gluconate were analyzed by using high performance liquid chromatography (HPLC) equipped with an Aminex HPX-87H column (Bio-Rad, USA). The cellulose production was quantified by measuring a weight after washing the cellulose solid produced in the flask with 0.1 N sodium hydroxide solution and distilled water, and freeze-drying the resultant. A gluconate yield was analyzed.

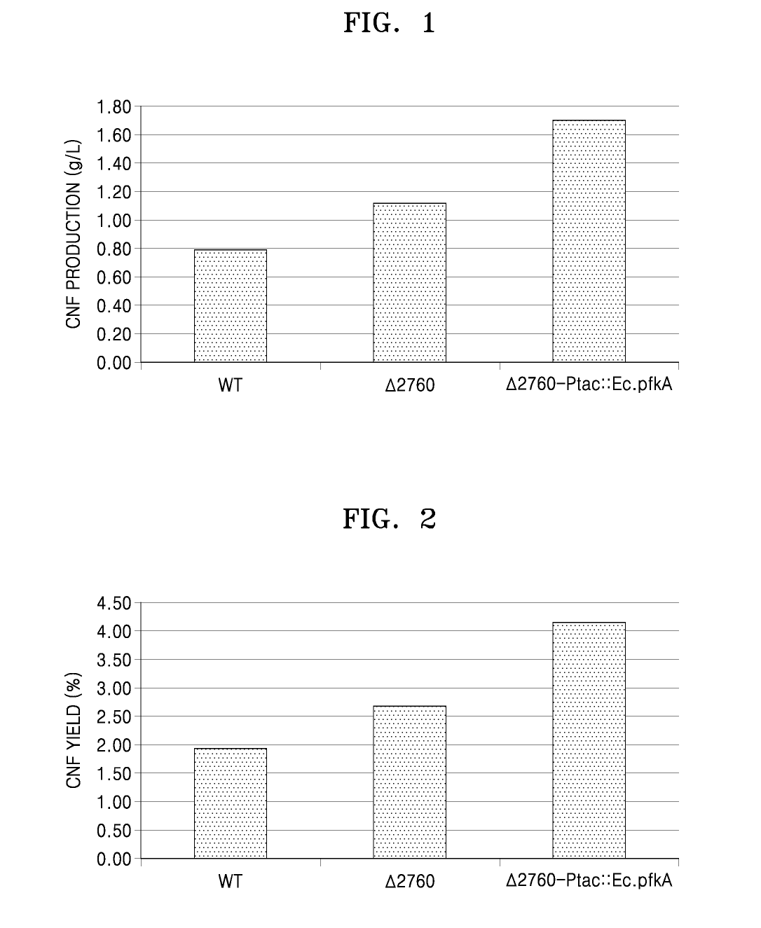

The results are shown in FIGS. 1 to 3. FIG. 1 shows CNF products from cultures which were obtained by culturing the K. xylinus strains. As shown in FIG. 1, when the pfkA gene was introduced into K. xylinus, the CNF production increased about 115% with respect to a wild-type strain. Table 1 illustrates the data shown in FIGS. 1 and 2.

TABLE 1

| | Glucose | Gluconate | | Gluconate | CNF |
|---|---|---|---|---|---|
| | consumption | production | CNF | yield | yield |
| | (g/L) | (g/L) | (g/L) | (%) | (%) |
| WT | 40.17 | 29.11 | 0.79 | 72.46 | 1.96 |
| Δ2760 | 41.65 | 31.71 | 1.12 | 76.13 | 2.68 |
| Δ2760- | 40.76 | 29.85 | 1.70 | 73.24 | 4.18 |
| Ptac::Ec.pfkA | | | | | |

FIG. 2 shows the yield of cellulose nanofibers (CNFs) obtained from the cultures prepared by culturing the K. xylinus strains. As shown in FIG. 2, when the pfkA gene was introduced into K. xylinus, the CNF yield increased about 113% with respect to a wild-type strain.

Also, the wild-type and recombinant strains were spread on HSD medium plates (5 g/L yeast extract, 5 g/L bacto peptone, 2.7 g/L Na2HPO₄, 1.15 g/L citric acid, and 20 g/L glucose) containing 20 g/L agar, and cultured at 30° C. for 3 days.

Starter fermentation was performed by adding 100 mL of HSD medium in a 250 mL flask, inoculating 3 loops of the microorganism, and culturing the resultant at 30° C. at 150 rpm for 20 hours.

Main fermentation was performed by using a 1.5 L bench-type fermentor (GX2-series, Biotron) system, a baffle was removed, and a stirring environment with enhanced vertical movement was formed by using a pitch-type impeller and a m icrosparger.

Operation conditions included an initial volume of 0.7 L, a temperature of 30° C., pH 5.0 (adjusted by using a neutralizing agent 3 N KOH (aq)), a stirring rate of 150 rpm, an airflow amount of 0.7 L/min, a medium, which was HS medium supplemented with 40 g/L glucose, and inoculation at a rate of 14%(v/v).

In the CMC-added environment, fermentation evaluation included adding Na_CMC 1.0%(w/v) to the same HS medium and changing the stirring rate to 250 rpm from the conditions described above. A CNF quantity was measured based on a weight after pre-treating the collected fermentation solution, that is, washing the collected fermentation solution with a 0.1 N NaOH (aq) solution at 90° C. for 2 hours.

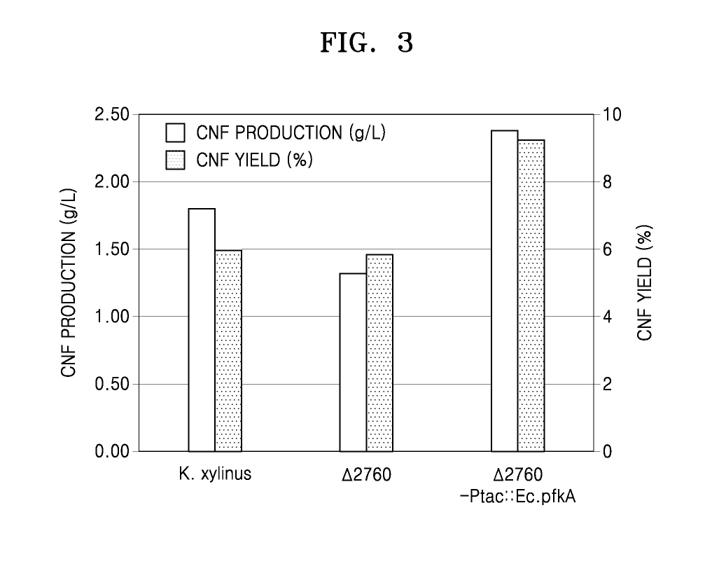

FIG. 3 shows CNF production and yield when the K. xylinus strains were cultured by fermentation. As shown in FIG. 3, when the pfkA gene was introduced into K. xylinus, CNF production increased about 32%, and the CNF yields increased about 55%, as compared with those of the control group. The yield is a percentage of the CNF weight produced with respect to a weight of glucose used. Table 2 illustrates the data shown in FIG. 3.

TABLE 2

| | CMC free fermentation | | |
|---|---|---|---|
| | Glucose | | |
| | consumption | CNF production | CNF yield |
| | (g/L) | (g/L) | (%) |
| WT | 29.70 | 1.80 | 5.95 |
| Δ2760 | 22.50 | 1.32 | 5.85 |
| Δ2760-Ptac::Ec.pfkA | 25.20 | 2.38 | 9.25 |

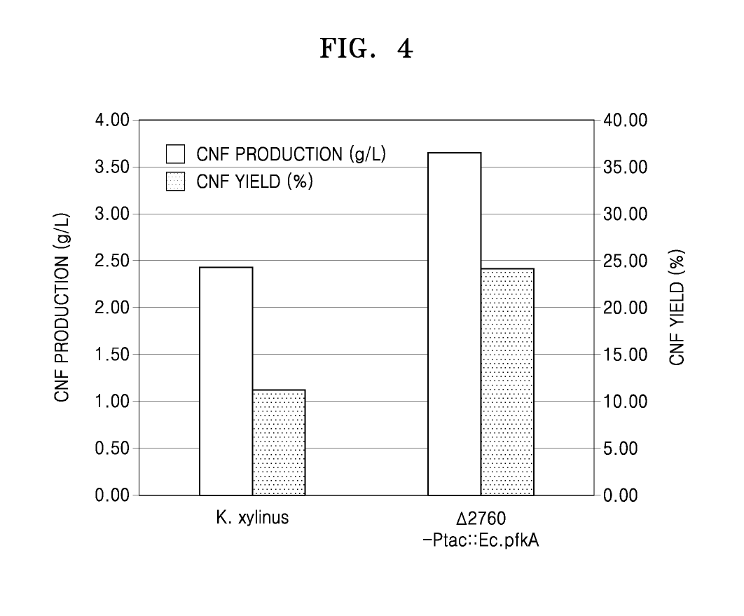

FIG. 4 shows CNF production and yield when the K. xylinus strains were fermented with CMC. As shown in FIG. 4, when the pfk gene was introduced into K. xylinus, the CNF production increased about 50%, and the CNF yields increased about 116%, as compared with those of the control group. Table 3 illustrates the data shown in FIG. 4.

TABLE 3

| | CMC added fermentation | | |
|---|---|---|---|
| | Glucose | | |
| | consumption | CNF production | CNF yield |
| | (g/L) | (g/L) | (%) |
| WT | 21.7 | 2.43 | 11.18 |
| Δ2760-Ptac::Ec.pfkA | 15.1 | 3.65 | 24.15 |

This indicates that the introduced exogenous pfkA phosphorylated fructose-6-phosphate of the strain into fructose-1,6-bisphosphate, and thus enhanced the glycolysis and influenced cellulose production.

### EXAMPLE 2

### Preparation of K. xylinus Including Polyphosphate Kinase Gene and Production of Cellulose

1. Preparation of Vector

Vectors used in this Example were prepared as follows.

A pMKO vector was prepared as follows. gapA promoter and rrnB terminator regions were amplified by using a pTSa-EX2 vector (SEQ ID NO: 20) as a template and a set of primers of SEQ ID NO: 21 and SEQ ID NO: 23 and a set of primers of SEQ ID NO: 26 and SEQ ID NO: 22. kan^{R} gene was amplified by using a pK19 mob-sacB vector (ATCC® 87098™) as a template and a set of primers of SEQ ID NO: 24 and SEQ ID NO: 25. Each of the PCR products was cloned by using an In-Fusion GD cloning kit (Takara) at the BamHI restriction site of a pUC19 vector (TAKARA) to prepare a pMKO vector.

A pMcodBA vector was prepared as follows. codBA gene (SEQ ID NO: 50) was amplified by using gDNA of E. coli as a template and a set of primers of SEQ ID NO: 27 and SEQ ID NO: 28. codBA gene is a gene needed to remove a marker gene used in the preparation of the strain and is a gene for negative selection. This PCR product was cloned by using an In-Fusion GD cloning kit (Takara) at the BglIl restriction site of the pMKO vector to prepare a pMcodBA vector.

A pMCT vector was prepared as follows. tac promoter and rrnB terminator regions were amplified by using a pTSa-EX11 vector (SEQ ID NO: 20) as a template and a set of primers of SEQ ID NO: 29 and SEQ ID NO: 30 and a set of primers of SEQ ID NO: 31 and SEQ ID NO: 32. Further, codBA and kan^{R} genes were amplified by using a pMcodBA vector as a template and a set of primers of SEQ ID NO: 33 and SEQ ID NO: 22. Each of the PCR products was cloned by using an In-Fusion GD cloning kit (Takara) at the BamHI restriction site of a pUC19 vector to prepare a pMCT vector.

A pMCT-(del)zwf vector was prepared as follows. Upstream and downstream regions of zwf gene were amplified by using gDNA of K. xylinus as a template and a set of primers of SEQ ID NO: 34 and SEQ ID NO: 35 and a set of primers of SEQ ID NO: 36 and SEQ ID NO: 37. Each of the PCR products was cloned by using an In-Fusion GD cloning kit (Takara) at the BamHI restriction site of a pMCT vector to prepare a pMCT-(del)zwf vector.

To prepare a vector for inserting polyphosphate kinase (PPK) gene, Silicibacter pomeroyi PPK3 gene (SEQ ID NO: 45), Silicibacter lacuscaerulensi PPK2 (SEQ ID NO: 47) and Rhodobacterales bacterium PPK2 gene (SEQ ID NO: 49) were synthesized (pUC57-Sp.ppk/Sl.ppk/Rb.ppk). The PPK3, PPK2 and PPK2 encode amino acid sequences of SEQ ID NOS: 44, 46, and 48, respectively. Each of the ppk genes was amplified by using a set of primers of SEQ ID NO: 38 and SEQ ID NO: 39, a set of primers of SEQ ID NO: 40 and SEQ ID NO: 41, and a set of primers of SEQ ID NO: 42 and SEQ ID NO: 43, and each resulting product was cloned by using an In-Fusion GD cloning kit (Takara) at the BglII restriction site of the pMCT-(del)zwf vector to prepare a pMCT-(del)zwf_Sp.ppk vector, a pMCT-(del)zwf_SI.ppk vector, and a pMCT-(del)zwf_Rb.ppk vector, respectively.

2. Transformation

In order to introduce Silicibacter pomeroyi PPK3 (Sp ppk), Silicibacter lacuscaerulensi PPK2 (SI ppk) and Rhodobacterales bacterium PPK2 (Rb ppk) genes, pMCT-(del)zwf_Sp.ppk, pMCT-(del)zwf_SI.ppk, and pMCT-(del)zwf_Rb.ppk were transformed into the SK3 strain which is a PFK gene-containing recombinant K. xylinus strain prepared in the clause 3 of Example 1 by the following transformation method to prepare SK3-(del)zwf_Sp.ppk, Sl.ppk, and Rb.ppk strains, respectively. A specific transformation procedure is as follows.

K. xylinus strain was spread on 2% glucose-supplemented HS medium containing 0.5% peptone, 0.5% yeast extract, 0.27% Na₂HPO₄, 0.15% citric acid, 2% glucose, and 1.5% bacto-agar, and cultured at 30° C. for 3 days. The strain was inoculated in 5 ml of HS medium supplemented with 0.2% cellulase (sigma), and then cultured at 30° C. for 2 days. A cell suspension thus cultured was inoculated in 100 ml of HS medium supplemented with 0.2% cellulase so that cell density (OD₆₀₀) was 0.04, and then the resultant was cultured at 30° C. so that cell density was 0.4 to 0.7. The cultured strain was washed with 1 mM of HEPES buffer, washed three times with 15% glycerol, and re-suspended with 1 ml of 15% glycerol to prepare competent cells.

K. xylinus DSM2325 M9 strain was spread on 2% glucose-supplemented HS plate, and then cultured at 30° C. for 3 days. The strain thus cultured was transferred to a 50 ml-falcon tube by using sterile water, followed by vortexing for 2 minutes. 1% cellulase (sigma, Cellulase from Trichoderma reesei ATCC 26921) was added thereto, and reaction was allowed at 30° C. and 160 rpm for 2 hours. Thereafter, the cultured strain was washed with 1 mM of HEPES buffer, washed three times with 15% glycerol, and re-suspended with 1 ml of 15% glycerol. 100 μl of the competent cells thus prepared were transferred to a 2-mm electro-cuvette, 3 μg of the plasmid was added thereto, and transformation was performed by electroporation (3.0 kV, 250 Ω, 25 μF). The cells were re-suspended in 1 ml of HS medium (2% glucose), transferred to a 14-ml round-bottom tube, and cultured at 30° C. and 230 rpm for 16 hours. The cultured cells were spread on an HS plate supplemented with 2% glucose, 1% ethanol, and 5 μg/ml of tetracycline or 50 μg/ml of kanamycin, and cultured at 30° C. for 5 days.

3. CNF Production

The K. xylinus strains introduced with respective vectors were streaked on HS plate supplemented with 2% glucose, 1% ethanol, and 5 μg/ml of tetracycline or 50 μg/ml of kanamycin, and cultured at 30° C. for 5 days. Then, the strains thus cultured were inoculated in 25 ml of HS medium supplemented with 5% glucose and 1% ethanol, and then cultured at 30° C., 230 rpm for 6 days. CNF thus produced was washed with 0.1N NaOH and distilled water at 60° C., and then freeze-dried to remove H₂O therefrom, followed by weighing. Glucose and gluconate were analyzed by HPLC.

CNF productions of the SK3 strains introduced with the respective ppk genes were compared, and as a result, the SK3 strains introduced with the respective ppk genes showed increased CNF productions, as compared with SK3 strain.

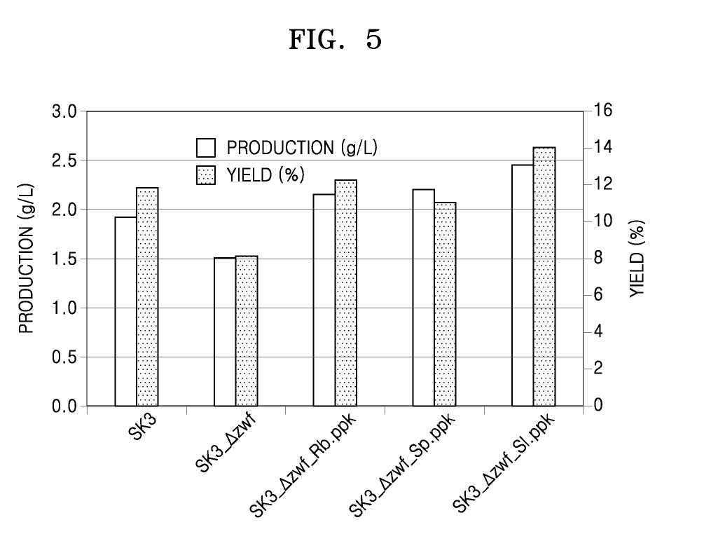

FIG. 5 shows results of comparing CNF productions of the SK3 strains which were introduced with the respective ppk genes. Table 4 shows the results of FIG. 5.

TABLE 4

| | Production (g/L) | | | | |
|---|---|---|---|---|---|
| | Glucose | | | CNF yield (%) | |
| Strain | consumed | Gluconate | CNF | CNF | Gluconate |
| SK3 | 16.20 | 10.21 | 1.92 | 11.85 | 63.05 |
| SK3_Δzwf | 18.55 | 7.92 | 1.51 | 8.14 | 42.70 |
| SK3_Δzwf_Rb ppk | 17.49 | 8.09 | 2.15 | 12.29 | 46.25 |
| SK3_Δzwf_Sp ppk | 19.93 | 3.95 | 2.21 | 11.09 | 19.84 |
| SK3_Δzwf_Sl ppk | 17.46 | 9.70 | 2.45 | 14.03 | 55.53 |

As shown in Table 4, the CNF production increased about 62.3%, and the CNF yield increased about 72.4% in SK3_Δzwf_SI ppk, as compared with SK3_Δzwf, indicating that introduced exogenous PPK enhanced supply of cofactors including ATP, GTP, UTP, CTP, or a combination thereof and influenced cellulose production.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism of the genus Komagataeibacter comprising a genetic modification that increases expression or activity of polyphosphate kinase (PPK).

2. The microorganism of claim 1, wherein the genetic modification increases expression of a gene encoding the polyphosphate kinase.

3. The microorganism of claim 1, wherein the genetic modification is an increase in the copy number of a gene encoding the polyphosphate kinase or a modification of an expression regulatory sequence of the gene encoding the polyphosphate kinase.

4. The microorganism of claim 1, wherein the polyphosphate kinase belongs to EC 2.7.4.1.

5. The microorganism of claim 1, wherein the polyphosphate kinase catalyzes both the forward and reverse reaction of converting NTP+ (phosphate)n to NDP+ (phosphate)n+1, and has higher catalytic activity for the reverse reaction than for the forward reaction.

6. The microorganism of claim 5, wherein the polyphosphate kinase has higher catalytic activity for conversion of NDP to NTP in a reaction using GDP, CDP, or UDP as a substrate, compared to using ADP.

7. The microorganism of claim 1, wherein the polyphosphate kinase is a polypeptide having a sequence identity of about 85% or more with an amino acid sequence of SEQ ID NO: 44, 46, or 48.

8. The microorganism of claim 1, wherein the polyphosphate kinase is a Silicibacter polyphosphate kinase or a Rhodobacterales polyphosphate kinase.

9. The microorganism of claim 1, wherein the microorganism has enhanced cellulose productivity as compared to a microorganism of the same type without the genetic modification that increases expression or activity of the polyphosphate kinase (PPK).

10. The microorganism of claim 1, further comprising a genetic modification that increases expression or activity of phosphofructose kinase (PFK).

11. The microorganism of claim 10, wherein the genetic modification is an increase of the copy number of a gene encoding the phosphofructose kinase or a modification of an expression regulatory sequence of the gene encoding the phosphofructose kinase.

12. The microorganism of claim 10, wherein the phosphofructose kinase belongs to EC 2.7.1.11.

13. The microorganism of claim 10, wherein the phosphofructose kinase is a polypeptide having a sequence identity of about 90% or more with an amino acid sequence of SEQ ID NO: 1.

14. The microorganism of claim 10, wherein the phosphofructose kinase is a Escherichia phosphofructose kinase, Bacillus phosphofructose kinase, Mycobacterium phosphofructose kinase, Zymomonas phosphofructose kinase, or Vibrio phosphofructose kinase.

15. The microorganism of claim 1, wherein the microorganism is Komagataeibacter xylinus.

16. A method of producing cellulose, the method comprising:
culturing the microorganism of claim 1 in a medium to produce cellulose; and
collecting the cellulose from a culture.

17. The method of claim 16, wherein the medium comprises ethanol or exogenous cellulose.

18. The method of claim 17, wherein the cellulose is carboxylated cellulose.

19. The method of claim 18, wherein the carboxylated cellulose is carboxy alkyl cellulose.

20. A method of producing a recombinant microorganism having enhanced cellulose productivity, the method comprising introducing a gene encoding polyphosphate kinase into a microorganism.

21. The method of claim 19, further comprising introducing a gene encoding phosphofructose kinase into the microorganism.


SEQUENCE LISTING (50 sequences)

SEQ ID NO: 1 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 2 - DNA, 963 bp, Escherichia coli
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 3 - DNA, 39 bp, Artificial Sequence
  Synthetic PFKA primer
cgtacccggggatccatgattaagaaaatcggtgtgttg

SEQ ID NO: 4 - DNA, 39 bp, Artificial Sequence
  Synthetic PFKA primer
gactctagaggatccttaatacagttttttcgcgcagtc

SEQ ID NO: 5 - DNA, 33 bp, Artificial Sequence
  Synthetic F1 forward primer
cggcgtagaggatcaggagcttatcgactgcac

SEQ ID NO: 6 - DNA, 28 bp, Artificial Sequence
  Synthetic F1 reverse primer
ccggcgtagagaatccacaggacgggtg

SEQ ID NO: 7 - DNA, 27 bp, Artificial Sequence
  Synthetic F2 forward primer
ctgtggattctctacgccggacgcatc

SEQ ID NO: 8 - DNA, 29 bp, Artificial Sequence
  Synthetic F2 reverse primer
aagggcatcggtcgtcgctctcccttatg

SEQ ID NO: 9 - DNA, 3576 bp, Artificial Sequence
  Synthetic pTSa-EX1 vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgttccgatcacctgtaacgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcaagaacatgtgagcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccttaaacgcctggttgctacgcctgaataagtgataataagcggatgaatggcagaaattc

SEQ ID NO: 10 - DNA, 40 bp, Artificial Sequence
  Synthetic primer
cttgatatcgaattcttctcatgtttgacagcttatcatc

SEQ ID NO: 11 - DNA, 36 bp, Artificial Sequence
  Synthetic primer
gggctgcaggaattcgaatttctgccattcatccgc

SEQ ID NO: 12 - DNA, 37 bp, Artificial Sequence
  Synthetic primer
cttgatatcgaattaggcctgtcatcgtctatatacg

SEQ ID NO: 13 - DNA, 42 bp, Artificial Sequence
  Synthetic primer
cgtgttgttcgaattcgatggatattcctccagtatcatgtg

SEQ ID NO: 14 - DNA, 32 bp, Artificial Sequence
  Synthetic primer
catcgaattcgaacaacacgccgatgtatgac

SEQ ID NO: 15 - DNA, 40 bp, Artificial Sequence
  Synthetic primer
acatgagaagaattgacagatccggtcagttcacattatc

SEQ ID NO: 16 - DNA, 39 bp, Artificial Sequence
  Synthetic primer
cagaaattcgaattgcgatcatcaccaaccaggaaattc

SEQ ID NO: 17 - DNA, 37 bp, Artificial Sequence
  Synthetic primer
gggctgcaggaattgggtatttcaggcggcagtaaag

SEQ ID NO: 18 - DNA, 40 bp, Artificial Sequence; identical to SEQ ID NO: 10
  Synthetic primer

SEQ ID NO: 19 - DNA, 36 bp, Artificial Sequence; identical to SEQ ID NO: 11
  Synthetic primer

SEQ ID NO: 20 - DNA, 3810 bp, Artificial Sequence
  Synthetic pTSa-EX2 vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgaacttcggcggcgcccgagcgtgaacagcacgggctgaccaacctgtgcgcgcggggcggttacgtgctggccgaagccgaaggcgcgcgacaggtcacgctgatcgccacgggacatgaggccatactggcactggcggcgcgcaaactgctgcgggacgcgggggttgcggcggctgtcgtctcccttccatgctgggaactgttcgccgtgcaaaaaatgacgtatcgtgccgccgtgctgggaacggcaccccggatcgggatcgaggccgcttcagggtttggatgggaacgatggcttggaacaggcgggctgtttgtcggtattgacggattcggggcgtcttacgcccccgaccggccagacagccctgccggcatcacgccggaacggatctgccacgacgcattgcggctggtccgcccccatgccgacgccctggttgaaaccgcgggaggaaacggcgcgccgcccgggatggcatcggtcgatgccagtgtgtgaaatgtcagaccttacggagaaaataagaaaaggacgagctattgattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcaagaacatgtgagcacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccttaaacgcctggttgctacgcctgaataagtgataataagcggatgaatggcagaaattc

SEQ ID NO: 21 - DNA, 32 bp, Artificial Sequence
  Synthetic primer
gactctagaggatccaacttcggcggcgcccg

SEQ ID NO: 22 - DNA, 33 bp, Artificial Sequence
  Synthetic primer
ggtacccggggatccgcaaaaaggccatccgtc

SEQ ID NO: 23 - DNA, 39 bp, Artificial Sequence
  Synthetic primer
aatattattgagatcttttcttattttctccgtaaggtc

SEQ ID NO: 24 - DNA, 33 bp, Artificial Sequence
  Synthetic primer
gaaaagatctcaataatattgaaaaaggaagag

SEQ ID NO: 25 - DNA, 31 bp, Artificial Sequence
  Synthetic primer
ccgccaggcatcagaagaactcgtcaagaag

SEQ ID NO: 26 - DNA, 26 bp, Artificial Sequence
  Synthetic primer
gttcttctgatgcctggcggcagtag

SEQ ID NO: 27 - DNA, 38 bp, Artificial Sequence
  Synthetic primer
aataagaaaagatcaatgtcgcaagataacaactttag

SEQ ID NO: 28 - DNA, 54 bp, Artificial Sequence
  Synthetic primer
atattattgagatcaaaggtctgacatttgatcatcaacgtttgtaatcgatgg

SEQ ID NO: 29 - DNA, 33 bp, Artificial Sequence
  Synthetic primer
cgactctagaggatcccggcgtagaggatcagg

SEQ ID NO: 30 - DNA, 46 bp, Artificial Sequence
  Synthetic primer
ccgccaggcaacctagatctggatcagctcggtacccaatcaatag

SEQ ID NO: 31 - DNA, 26 bp, Artificial Sequence
  Synthetic primer
agatctaggttgcctggcggcagtag

SEQ ID NO: 32 - DNA, 34 bp, Artificial Sequence
  Synthetic primer
gggcgccgccgaagttgcaaaaaggccatccgtc

SEQ ID NO: 33 - DNA, 26 bp, Artificial Sequence
  Synthetic primer
gcctttttgcaacttcggcggcgccc

SEQ ID NO: 34 - DNA, 34 bp, Artificial Sequence
  Synthetic primer
cgactctagaggatccgtcgagcagggtttcctg

SEQ ID NO: 35 - DNA, 34 bp, Artificial Sequence
  Synthetic primer
ctctacgccgggatctgccgatgggtttttccag

SEQ ID NO: 36 - DNA, 35 bp, Artificial Sequence
  Synthetic primer
gcctttttgcggatctgaaaagtcgagcgagatcg

SEQ ID NO: 37 - DNA, 33 bp, Artificial Sequence
  Synthetic primer
cggtacccggggatcccagacagtccctcatcg

SEQ ID NO: 38 - DNA, 31 bp, Artificial Sequence
  Synthetic primer
agctgatccagatctatgacgctgccattcg

SEQ ID NO: 39 - DNA, 32 bp, Artificial Sequence
  Synthetic primer
aggcaacctagatctggcatcccaaatatccg

SEQ ID NO: 40 - DNA, 32 bp, Artificial Sequence
  Synthetic primer
agctgatccagatctatgaaccgtaacggcag

SEQ ID NO: 41 - DNA, 32 bp, Artificial Sequence
  Synthetic primer
aggcaacctagatctcgcatcccaaatatccg

SEQ ID NO: 42 - DNA, 32 bp, Artificial Sequence
  Synthetic primer
agctgatccagatctatgacccgtactgctcc

SEQ ID NO: 43 - DNA, 32 bp, Artificial Sequence
  Synthetic primer
aggcaacctagatctggcatcccaaatttccg

SEQ ID NO: 44 - protein, 301 aa, Silicibacter pomeroyi
MNRNGSTKDPRRMTGAATGEISRYFNDKAPKDIRRAIEKADKDDILSTTYPYDAEMTAKDYRAQMEALQIELVKLQAWIKQSGARVALLFEGRDAAGKGGTIKRFRENLNPRGARVVALSKPTEAERSQWYFQRYIQHLPSAGELVFYDRSWYNRGVVEHVFGWCDEEQRERFFRQVMPFEHDLVDDGIHLFKFWLNVGRAEQLRRFHDRERDPLKQWKLSPVDIAGLDKWEAYTTAISQTLTRSHSDRAPWTVIRSDDKKRARLAAIRTVLSGIDYDNKDRAAVGQPDAAICGGPDIWDA

SEQ ID NO: 45 - DNA, 903 bp, Silicibacter pomeroyi
atgaaccgtaacggcagcactaaggaccctcgtcgtatgacgggtgcagcaactggtgagatcagccgttacttcaacgacaaagccccgaaagacatccgccgtgcaatcgagaaggcagacaaagacgacatcctgtccaccacctacccgtacgacgcagagatgaccgctaaagactaccgtgcgcaaatggaagctctgcagatcgaactggtcaaactgcaggcgtggatcaagcagagcggcgcacgtgtagcactgctgttcgaaggtcgtgacgctgctggtaaaggtggtactatcaaacgtttccgtgagaacctgaacccgcgtggcgctcgtgtcgtggctctgtctaaaccaaccgaagctgaacgtagccagtggtacttccagcgttacatccagcacctgccatccgctggtgaactggtattctacgaccgctcttggtataaccgcggcgtggtggaacacgtgtttggttggtgcgacgaagaacagcgtgaacgtttcttccgccaggttatgccgttcgaacacgacctggttgacgacggtatccacctgttcaaattttggctgaatgtaggccgcgcggaacagctgcgtcgctttcatgaccgcgaacgcgatccgctgaaacagtggaaactgtccccggttgatatcgcgggtctggataaatgggaagcctataccacggcgatctcccaaaccctgacccgttcccattctgatcgtgccccgtggactgttattcgttctgatgataagaaacgtgcgcgcctggccgcgattcgcaccgttctgtctggcattgattatgataacaaagatcgcgcggcggttggccagccggatgccgctatttgtggcggcccggatatttgggatgcg

SEQ ID NO: 46 - protein, 289 aa, Silicibacter lacuscaerulensis
MTRTAPGAINDYFRNHAPKDVRQAIENAGKDDILNPSYPYSERMKGKVYDRHMDALQIELVKMQSWVKETGQRIAIIFEGRDAAGKGGTIKRFRENLNPRGARNVALAKPTEAERSQWYFQRYVQHLPSAGEIVFFDRSWYNRGVVENVFGFCTPEERERFFRQVLPLEHGFVNDGIRLFKFWLNVGRAEQLNRFLARENDPLKQWKLSPIDIAGLGKWDEYTQSISETLTRSHSDICPWTIVRSDDKKRARIATIQTVLAALDYDEKDPDVVGTPDPNIAGGPEIWDA

SEQ ID NO: 47 - DNA, 867 bp, Silicibacter lacuscaerulensis
atgacccgtactgctcctggtgcaatcaacgattacttccgtaaccacgcgcctaaagacgtgcgtcaggcaatcgagaacgcaggtaaagacgacatcctgaacccaagctacccatactccgaacgcatgaaaggcaaagtctacgaccgccacatggacgctctgcaaatcgaactggtcaagatgcagagctgggtgaaagaaacgggccagcgtatcgcaatcatcttcgaaggtcgcgacgcagcaggtaaaggtggtaccatcaagcgcttccgtgagaacctgaacccgcgtggtgctcgtaatgtggctctggctaaaccgactgaagctgaacgttcccaatggtacttccagcgttacgtgcagcacctgccgtctgcgggtgaaattgtattcttcgaccgtagctggtataaccgtggtgtggttgagaacgtattcggcttctgcaccccggaagaacgtgaacgtttcttccgtcaggttctgccgctggaacatggttttgttaacgacggcatccgcctgtttaaattttggctgaacgttggccgcgccgaacagctgaaccgttttctggcccgtgagaacgacccgctgaaacagtggaaactgtccccgattgacatcgcgggtctgggcaaatgggatgaatatacgcagtctatctccgaaaccctgacccgttctcattctgacatctgtccgtggaccatcgtacgctctgatgataagaaacgcgcgcgcattgcgaccattcagactgttctggcggcgctggattatgatgagaaagatccggatgttgttggcactccggatccgaatattgccggcggcccggaaatttgggatgcc

SEQ ID NO: 48 - protein, 289 aa, Unknown
  Rhodobacterales
MTLPFDGAISRYYETGAPEEIRKAIQTADKDEIITPSYPHRERMARKTYEAELEALQIELVKMQAWVKASGARIAIVLEGRDAAGKGGTIKRFRENLNPRGARVVALSKPSEEEQSQWYFQRYIQHLPSGGEIVFFDRSWYNRGVVEKVFGFCTDDQRERFFHQVKGFEQALVDDGVKLFKFWLNVGRAEQLRRFLKRESDPLKQWKLSPIDVKGLEKWDEYTAAISETLQRSHCPEAPWTIVRSDGKRRARLAAIRAVLSGIDYDNKDAKAIGALDTEICGGPDIWDA

SEQ ID NO: 49 - DNA, 867 bp, Unknown
  Rhodobacterales
atgacgctgccattcgatggtgctatcagccgttactacgaaacgggtgcacctgaagaaatccgtaaggctatccagactgctgacaaagacgagatcatcaccccatcctacccgcaccgtgaacgtatggcacgtaagacctacgaagctgaactggaagcgctgcagatcgaactggtcaagatgcaggcatgggtcaaggctagcggtgctcgtatcgctatcgtactggaaggtcgtgacgctgcaggtaaaggtggtactatcaaacgcttccgtgagaacctgaacccgcgtggtgcacgtgtagttgcactgtctaaaccgtctgaagaggagcagtcccagtggtacttccagcgctacatccagcacctgccgtctggtggcgaaatcgtattcttcgaccgttcctggtataaccgcggtgtggtggagaaagtgttcggtttctgcaccgacgaccagcgtgaacgctttttccaccaggtgaaaggcttcgaacaggcactggttgacgacggcgttaaactgtttaaattttggctgaatgttggccgcgcggaacagctgcgccgttttctgaaacgcgaatccgacccgctgaaacaatggaaactgtccccgattgacgttaaaggcctggagaaatgggatgaatataccgccgcgatcagcgaaaccctgcaacgcagccattgcccggaagcgccgtggactatcgttcgttctgatggcaaacgtcgcgcgcgtctggcggccattcgtgccgttctgtctggcattgattatgataacaaagatgcgaaagcgattggcgcgctggataccgaaatttgtggcggcccggatatttgggatgcc

SEQ ID NO: 50 - DNA, 2533 bp, Escherichia coli
atgtcgcaagataacaactttagccaggggccagtcccgcagtcggcgcggaaaggggtattggcattgacgttcgtcatgctgggattaaccttcttttccgccagtatgtggaccggcggcactctcggaaccggtcttagctatcatgatttcttcctcgcagttctcatcggtaatcttctcctcggtatttacacttcatttctcggttacattggcgcaaaaaccggcctgaccactcatcttcttgctcgcttctcgtttggtgttaaaggctcatggctgccttcactgctactgggcggaactcaggttggctggtttggcgtcggtgtggcgatgtttgccattccggtgggtaaggcaaccgggctggatattaatttgctgattgccgtttccggtttactgatgaccgtcaccgtcttttttggcatttcggcgctgacggttctttcggtgattgcggttccggctatcgcctgcctgggcggttattccgtgtggctggctgttaacggcatgggcggcctggacgcattaaaagcggtcgttcccgcacaaccgttagatttcaatgtcgcgctggcgctggttgtggggtcatttatcagtgcgggtacgctcaccgctgactttgtccggtttggtcgcaatgccaaactggcggtgctggtggcgatggtggcctttttcctcggcaactcgttgatgtttattttcggtgcagcgggcgctgcggcactgggcatggcggatatctctgatgtgatgattgctcagggcctgctgctgcctgcgattgtggtgctggggctgaatatctggaccaccaacgataacgcactctatgcgtcgggtttaggtttcgccaacattaccgggatgtcgagcaaaaccctttcggtaatcaacggtattatcggtacggtctgcgcattatggctgtataacaattttgtcggctggttgaccttcctttcggcagctattcctccagtgggtggcgtgatcatcgccgactatctgatgaaccgtcgccgctatgagcactttgcgaccacgcgtatgatgagtgtcaattgggtggcgattctggcggtcgccttggggattgctgcaggccactggttaccgggaattgttccggtcaacgcggtattaggtggcgcgctgagctatctgatccttaacccgattttgaatcgtaaaacgacagcagcaatgacgcatgtggaggctaacagtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttga